(19) United States
(12) Patent Application Publication
(10) Pub. No.: US 2024/0261220 A1
(43) Pub. Date: Aug. 8, 2024

(54) APTAMER FUNCTIONALIZED LIPOSOMES FOR DELIVERY OF OCULAR DRUG FOR EYE DISEASES

(71) Applicants: JUEWEN LIU, Waterloo (CA); YIBO LIU, Waterloo (CA); CENTRE FOR EYE AND VISION RESEARCH LIMITED, Shatin (HK); THE HONG KONG POLYTECHNIC UNIVERSITY, Kowloon (HK)

(72) Inventors: MAN-SAU WONG, Kowloon (HK); KA-YING WONG, Kowloon (HK); JUEWEN LIU, Waterloo (CA); YIBO LIU, Waterloo (CA)

(21) Appl. No.: 18/106,205

(22) Filed: Feb. 6, 2023

(51) Int. Cl. A61K 9/127; A61K 38/13; A61K 47/54; A61P 27/02

(52) U.S. Cl. CPC A61K 9/127; A61K 38/13; A61K 47/549; A61P 27/02

(57) ABSTRACT

Provided herein are aptamer functionalized liposomes for delivery of ocular drug for eye diseases (such as dry eye disease), products comprising the same and method for preventing and/or treating eye diseases using the same. The aptamer functionalized liposomes, product and method of the present application can be used to effectively delivering ocular drug with extended retention time and high protective effect.

### TECHNICAL FIELD

The present disclosure relates generally to the field of medicine. More particularly, it concerns aptamer functionalized liposomes for delivery of ocular drug for treating dry eye diseases.

### STATEMENT REGARDING SEQUENCE LISTING

The sequence listing associated with this application is provided in text format in lieu of a paper copy and is hereby incorporated by reference into the specification. The name of the XML file containing the sequence listing is Sequencelisting. The XML file is 11,286 bytes; was created on Feb. 3, 2023.

### BACKGROUND OF THE INVENTION

Ocular drug delivery has been a challenging topic as human eyes have static (corneal epithelium, corneal stroma, corneal endothelium, blood-aqueous barrier) and dynamic (tear dilution, conjunctival barrier, and retinal-blood barrier) barriers to prevent external substances, including therapeutic molecules, from getting into the eyes. In the past two decades, the rapid development of ocular drug delivery has provoked newer therapeutic interventions, like liposomes, micelles, polymer nanoparticles, drug-eluting contact lenses, and ocular inserts, to improve drug absorption, drug concentration at the target site, drug retention time and biocompatibility.^{[1]}

Recently, functionalization of liposomes with aptamers broadened the use of liposomes in targeted drug delivery.^{[2]} Aptamers are short single-stranded RNA or DNA oligonucleotides that specifically bind to their targets due to their unique 3-dimensional structures. Aptamers have been demonstrated to have less toxicity and immunogenicity compared to other targeting ligands, like antibodies.^{[2, 3]} Pegaptanib, an RNA aptamer targeting vascular endothelial growth factor, is an FDA-approved drug for age-related macular degeneration.^{[4]} With merits of aptamer in easy synthesis, chemical modification, and lipid conjugation, aptamer-functionalized liposomes have been shown to have promising targeted-drug delivery in cancer therapy, which achieved better bioavailability, retention time, and site-specific delivery of drugs.^{[5]}

Cyclosporine A (CsA), a hydrophobic endecapeptide molecule, is an FDA-approved ocular drug for dry eye diseases (DED). CsA functions as an immunosuppressive agent to reduce inflammation and interfere with tear production.^{[6]} However, CsA possesses low solubility in aqueous solutions which makes it difficult to be delivered safely and effectively into the eyes.^{[6, 7]} Current oil-based CsA eye drops have low bioavailability and require multiple administration. Aqueous nanomicellar CsA solution was invented to improve the delivery of CsA into the eye. However, the major route of aqueous CsA absorption in the eyes is through the conjunctiva and sclera and is mainly delivered to the posterior tissues.^{[7]} Also, the target site of DED does not include the posterior segment of the eye.^{[8]}

There is still a great need for targeted delivery of DED treatment drug (such as CsA) to the cornea, the disease site of DED, and for increasing the retention time in the cornea and increasing the bioavailability of the drug in treatment of DED.

### SUMMARY OF THE INVENTION

Disclosed herein are novel site-specific drug delivery tool by using aptamer-functionalized liposomes. Disclosed herein are also methods for the treatment of DED using the aptamer-functionalized liposomes.

According to one aspect, the present disclosure provides aptamer functionalized liposomes loaded with active ingredient(s) for preventing and/or treating eye disease.

According to a further aspect, the present disclosure provides a product for preventing and/or treating eye disease comprising the aptamer functionalized liposomes loaded with active ingredient(s) for preventing and/or treating eye disease, and a pharmaceutically or physiological acceptable carrier and/or excipient.

According to a further aspect, the present disclosure provides a method for preventing and/or treating eye disease in a subject in need of the prevention and/or treatment comprising administering an effective amount of a aptamer functionalized liposomes loaded with active ingredient(s) for preventing and/or treating eye disease or a product comprising the same to the eye(s) of the subject.

According to a further aspect, the present disclosure provides a method for targeted drug delivery to the corneal epithelium of a subject in need thereof comprising administering an effective amount of a aptamer functionalized liposomes loaded with active ingredient(s) for preventing and/or treating eye disease or a product comprising the same to the eye(s) of the subject.

In some embodiments, the aptamer is a mucin-targeting aptamer. In some embodiments, the eye disease is a noncancerous disease, such as a dry eye disease.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1(A) to (D): Schematic diagram for the preparation of the CsA-loaded liposome conjugated with mucin-1 aptamer and the CsA delivery and release process in cells.

FIG. 1(A): the structural formula of Cyclosporin A (CsA);

FIG. 1(B): CsA-loaded liposome;

FIG. 1(C): Mucin-1 aptamer linked with cholesterol (upper) which further conjugated with CsA-loaded liposome to form CsA-loaded liposome conjugated with mucin-1 aptamer (lower); and

FIG. 1(D): targeted delivery and release of CsA-loaded liposome conjugated with mucin-1 aptamer in cells.

FIGS. 2(A) to (F): Characterization of the synthesized liposome.

FIG. 2(A): the mean particle size of synthesized liposomes with different CsA loading analyzed by DLS;

Data were shown as mean±SEM and analyzed using One-Way ANOVA analysis; *P<0.05, **P<0.01, ***P<0.001 compared with 0% group;

FIG. 2(B): Size distribution of liposome with different CsA loading by intensity;

FIG. 2(C): the Zeta potential of synthesized liposomes with different CsA loading analyzed by DLS;

FIG. 2(D): UV spectrum of CsA at various concentrations;

FIG. 2(E): Calibration curve of CsA at various concentrations; and

FIG. 2(F): the amount of free aptamer after aptamer conjugation to the liposome;

Data were shown as mean±SEM and analyzed using One-Way ANOVA analysis; *P<0.05, **P<0.01, ***P<0.001 compared with 0% group.

FIG. 3: Cytotoxicity of the synthesized liposome. DED condition in HCECs was induced by hyperosmotic pressure. The cells were treated with free CsA, drug-free liposomes, and CsA-loaded liposomes at different concentrations for 24 hours. MTS assay was performed to measure the cell viability.

Data were shown as mean±SEM and analyzed using One-Way ANOVA analysis. ***P<0.001 compared with DED group.

FIGS. 4(A) to (B): Cellular uptake of the synthesized liposome.

FIG. 4(A): Fluorescence images of HCECs incubated with liposomes for 4 hours; and

FIG. 4(B): Fluorescence images of HCECs incubated with liposomes for 4 hours followed by 20-hours incubation in culture medium after wash.

The cell nucleus was stained with DAPI (first row) to give blue fluorescence and the red fluorescence (second row) was from rhodamine-labelled PE (Rhod PE). The overlay of two fluorescence was captured (last row). Scale bar: 20 μm; and “Rho”: Rhod PE.

FIGS. 5(A) to (D): Gene expressions of dry eye gene markers. The model cells were treated with free CsA, drug-free liposomes, and CsA-loaded liposomes at different concentrations for 4 hours followed by 20-hour incubation in the culture medium after wash. RT-PCR was performed to measure the mRNA expressions of FIG. 5(A) IL-6, FIG. 5(B) IL-8, FIG. 5(C) IL-1B, and FIG. 5(D) MMP-1.

Data were normalized with GAPDH and relative to control. Data were shown as mean±SEM and analyzed using One-Way ANOVA analysis. *P<0.05, **P<0.01, ***P<0.001 compared with DED group.

FIGS. 6(A) to (B): Cell permeability of fluorescein.

FIG. 6(A) Fluorescence images of treated HCECs incubated with 1 mM fluorescein for 5 minutes. 4 hours followed by 20-hour incubation in the culture medium after wash. The cell nucleus was stained with DAPI (first row) to give blue fluorescence and the green fluorescence (second row) was from fluorescein (FITC). The overlay of two fluorescence was captured (last row). Scale bar: 20 μm; and

FIG. 6(B): Quantification of fluorescent intensity.

Data were shown as mean±SEM and analyzed using One-Way ANOVA analysis. ***P<0.001 compared with DED group; \hat{ }\hat{ }P<0.01 compared with CsA-loaded liposome conjugated with S2.2.

### DETAILED DESCRIPTION OF THE INVENTION

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

Traditional eye drops are convenient to use but their effectiveness is limited by their poor retention time and bioavailability in the eyes due to ocular barriers. Strategies for enhancing ocular drug delivery are demanding. In the present study, a mucin-targeting aptamer-functionalized liposome was constructed and loaded with Cyclosporin A, a common ocular drug in eye drops to treat dry eye diseases. Drug encapsulation decreased liposome size and did not change the surface potential of liposomes. Around 90% of the aptamer was successfully conjugated to liposomes. The cytotoxicity, anti-inflammatory effects, tight junction regulation, and retention time of liposomes in the human corneal epithelial cells were evaluated under dry eye conditions. The results suggested that the mucin-1 aptamer functionalized liposomes were more efficient as nanocarriers than the non-functionalized liposome and drug-free liposome by restoring the inflammation level and retaining in cells for extended time.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described.

Reference to “the disclosure” and “the invention” and the like includes single or multiple aspects taught herein; and so forth. Aspects taught herein are encompassed by the term “invention”.

As used herein, the term “a” or “an” is intended to mean “one or more” (i.e., at least one) of the grammatical object of the article. Singular expressions, unless defined otherwise in contexts, include plural expressions. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The use of “or” means “and/or” unless stated otherwise.

As used herein, unless otherwise noted, the term “comprise”, “include” and “including” will be understood to imply the inclusion of a stated step or element or group of steps or elements but not the exclusion of any other step or element or group of steps or elements which do not affect the end result. The terms “comprising”, “comprises” and “comprised” may also include the term “consisting essentially of” and “consisting of”.

The phrase “consisting essentially of” indicates that the listed elements are required or mandatory, but that other elements are optional and may or may not be present depending upon whether or not they materially affect the activity or action of the listed elements. The phrase “consisting of” is meant to include, and is limited to, whatever follows the phrase “consisting of.” Thus, the phrase “consisting of” indicates that the listed elements are required or mandatory and that no other elements may be present.

The term “isolated” refers to a material that is substantially or essentially free from components that normally accompany it in its native state. The material can be a cell or a macromolecule such as a protein or nucleic acid. For example, an “isolated cell”, as used herein, refers to a cell, which has been purified from the cells in a naturally-occurring state.

### Liposome

The present application utilizes liposomes within which active ingredients for preventing and/or treating eye diseases is carried and to which an aptamer (such as mucin-targeting-aptamer) is conjugated for functionalizing the liposomes such as for extending the retention time of the liposomes loading with the active ingredients and for improving the anti-inflammation effect.

Liposomes formed from various materials can be used in the present application as long as the selected materials are compatible to the active ingredients and can be conjugated to the aptamer. The materials for forming the liposome may include but not limited to lipids having an anionic, cationic or zwitterionic hydrophilic head group. Some phospholipids are anionic whereas others are zwitterionic or cationic. Suitable classes of phospholipid include, but are not limited to, phosphatidylethanolamines (PE), phosphatidylcholines, phosphatidylserines, and phosphatidyl-glycerols. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are 1,2-Dioleoyl-sn-glycero-3-phosphocholine (DOPC), 1,2-dioleoyl-sn-glycero-3-phosphatidylethanolamine (DOPE), 1,2-dihexadecanoyl-rac-glycero-3-phosphocholine (DPPC), 1,2-distearoyl-sn-glycero-3-phosphocholine (DSPC), dodecylphosphocholine, and 1,2-diphytanoyl-sn-glycero-3-phosphoethanolamine (DPyPE). The lipids can be saturated or unsaturated. The use of at least one unsaturated lipid for preparing liposomes is preferred. If an unsaturated lipid has two tails, one or both tails can be unsaturated.

Liposomes of the invention can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMG (anionic, saturated). Where a liposome of the invention is formed from lipids, a proportion of those lipids may be PEGylated.

In some embodiments, the liposomes in the present application are formed from DOPC which is the dominant component of the phospholipid bilayer of cell membranes and Rhod PE.

In general, liposomes can be classified into three types by sizes: small unilamellar vesicles (SUV, 15-100 nm in diameter), large unilamellar vesicles (LUV, 100-1000 nm in diameter), and giant unilamellar vesicles (GUV, 1-5 μm in diameter). Small unilamellar vesicles and large unilamellar vesicles have a larger specific surface area which might increase solubility, enhance the bioavailability, and enable accurate targeting of the encapsulated material to a greater extent. Liposomes of the invention are ideally SUV and LUVs with a diameter in the range of 60˜180 nm, and preferably in the range of 80˜160 nm.

The liposomes of the present application are loaded with active ingredients for preventing and/or treating eye diseases. The active ingredients may comprise but not limited to those for preventing and/or treating dry eye disease (DED), glaucoma, cataract, keratitis, conjunctivitis, mydriasis, optic neuritis, diabetic retinopathy, refractive error, fundus hemorrhage, strabismus, night blindness, macular degeneration, pterygium, ocular calculi, lacrimal sac.

In some embodiments, the active ingredient for preventing and/or treating eye disease is one or more selected from the group consisting of: polypeptide agent (such as cyclosporine A, defensins, interferons, epidermal growth factor), anti-glaucomatous agent (such as β-blockers), antibiotics (such as β-lactam, aminoglycosides, macrolide), antibacterial (such as quinolone, imidazole), antiviral agent (such as nucleosides), anti-inflammation agent (such as steroidal anti-inflammatory drugs, non-steroidal anti-inflammatory drugs).

The loading content of active ingredient may be varied depends on the type of the active ingredient, the material used to form the liposome, the ways used to load the active ingredient to the liposomes. Usually, the percentage loading content of the active ingredient in the liposome is about 0.001˜40 wt % based on the total weight of the liposome. In some embodiments, the encapsulation efficiency of the active ingredient in the liposome is about 50˜100%.

### Aptamer Functionalized Liposome

As used herein, the term “aptamer” refers to short single-stranded RNA or DNA oligonucleotides or XNA (xeno nucleic acid) that specifically bind to their targets due to their unique 3-dimensional structures. Aptamers have been demonstrated to have less toxicity and immunogenicity compared to other targeting ligands, like antibodies. The K_{d} of an aptamer suitable for use in the present application to the target thereof in cornea is about 0.1 nM to 300 nM. In some embodiments, the aptamer comprises 18-60 nucleotides, preferably 20-25 nucleotides.

The aptamer(s) binding to cornea may include but not limited to mucin-targeting aptamer. As used herein, “mucin-targeting-aptamers” are aptamers with high binding specificity and affinity to Mucin, especially Mucin-1. In some embodiments, the K_{d} of the mucin-targeting-aptamer to mucin-1 is about 0.1 nM to 50 nM.

Most of the current aptamer-functionalized deliveries are targeting cancers. There is no research about employing aptamers in ocular delivery, let along employing the aptamers to functionalize liposomes loaded with active ingredients for preventing and/or treating eye diseases.

Aptamers can be screened and selected by methods known in the art, such as by systematic evolution of ligands by exponential enrichment (SELEX), from nucleic acid molecular library. Aptamers suitable to be used in the present application can be selected by methods known in the art or can be aptamers known in the art. For example, mucin-targeting-aptamers already known in the art or screened and obtained via known methods can be used in the present application. As a particular example, aptamer S2.2 as indicated in the Example portion of the present application is a preferred aptamer for the present invention. In some embodiments, the mucin-targeting aptamer is one comprising a nucleotide sequence shown in SEQ ID NO: 1.

In some embodiments, to facilitate the conjugation of the aptamer to the liposome, the aptamer may have a lipophilic part linked at one or both ends thereof. In some embodiments, the lipophilic part is cholesterol such that the aptamer can insert into the liposomes.

The aptamer can be conjugated to the liposome by conventional method known in the art, such as by co-incubation of a suspension comprising the aptamer and a solution comprising the liposome. A liposome may have tens to hundreds aptamers inserted therein, for example about 50-500 aptamers per liposome.

The aptamer functionalized liposome loading with active ingredients of the present application has remarkably improved, even synergic effect in prolonging the retention time of the drug which results in prolonged drug effect and improved protection effect (such as in anti-inflammation).

### Pharmaceutical Compositions

The therapeutic or prophylactic liposomes produced by the materials and methods of the present disclosed can be used to prepare pharmaceutical compositions for the treatment or prevention of eye disease. A pharmaceutical composition of the present disclosure typically includes a therapeutically or prophylactically effective amount of a functionalized liposome and a pharmaceutically acceptable carrier.

As used herein, “pharmaceutically acceptable carrier” can be any solvent, dispersion medium, antibacterial or antifungal agent, isotonic or absorption delaying agent, or the like. The use of such media and agents for pharmaceutically active substances is well known in the art. Supplementary active ingredients can also be incorporated into a pharmaceutical composition of the present invention.

Administration of a pharmaceutical composition of the present invention can be via any common route for preventing or treating eye disease. Solutions of interest can be prepared in water suitably mixed with a surfactant, such as hydroxypropylcellulose. Dispersions can also be prepared in glycerol, liquid polyethylene glycols, or mixtures thereof, or in oils. Under ordinary conditions of storage and use, these preparations can contain a preservative to prevent the growth of microorganisms.

Suitable pharmaceutical carriers include, but are not limited to, solvents or dispersion media containing, for example, water, ethanol, polyol (e.g., glycerol, propylene glycol, and liquid polyethylene glycol, and the like), or vegetable oils. The proper fluidity can be maintained, for example, by the use of a coating, such as lecithin, by the maintenance of the required particle size in the case of dispersion, or by the use of surfactants. The prevention of the action of microorganisms can be brought about by various antibacterial or antifungal agents, for example, parabens, chlorobutanol, phenol, sorbic acid, thimerosal, or the like. In many cases, it will be preferable to include isotonic agents, for example, sugars or sodium chloride. Prolonged absorption of the injectable compositions can be brought about by the use of agents delaying absorption, for example, aluminum monostearate and gelatin.

Sterile solutions can be prepared by incorporating a therapeutic or prophylactic liposomes in the required amount in an appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients.

Upon formulation, compositions or solutions can be administered in a manner compatible with the dosage formulation and in such amount as is therapeutically or prophylactically effective. The dosage regimen can be determined by the attending physician based on various factors such as the action of the protein, the site of pathology, the severity of disease, the patient's age, sex and diet, the severity of any inflammation, time of administration, and other clinical factors.

As compared to conventional ocular drug, the aptamer (such as mucin-targeting-aptamer) functionalized liposome and pharmaceutical composition comprising the same at least have the following advantages: targeted drug delivery to the corneal epithelium; prolonged retention time; comparable efficacy at a lower dose; prevention of drug metabolism; increased absorption through endocytosis; reduced clearance by mucus; less administration time; and cheaper to manufacture. Hence, the present application provides a better tool for ocular treatment.

### Specific Embodiments

Further embodiments of the present invention are described again in the following. The present invention in particular also provides for the following items:
1. Aptamer functionalized liposomes loaded with active ingredient(s) for preventing and/or treating eye disease.
2.1. The functionalized liposomes of item 1, wherein the mean particle size of the liposomes is about 10˜1000 nm.

The functionalized liposomes of item 1, wherein the mean particle size of the liposomes is about 50˜500 nm.

The functionalized liposomes of item 1, wherein the mean particle size of the liposomes is about 60˜200 nm.
2.2. The functionalized liposomes of item 1, wherein the zeta potential of the liposome is about 0˜5 mV.
2.3. The functionalized liposomes of item 1, wherein the liposomes are prepared from lipids have an anionic, cationic or zwitterionic hydrophilic head group.
3. The functionalized liposomes of item 1, wherein the liposomes are prepared from phospholipid selected from phosphatidylethanolamines (PE), phosphatidylcholines, phosphatidylserines, and phosphatidyl-glycerols, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), DPPC, DSPC, DOPC, dodecylphosphocholine, 1,2-dioleoyl-sn-glycero-3-phosphatidylethanolamine (DOPE), and 1,2-diphytanoyl-sn-glycero-3-phosphoethanolamine (DPyPE).
4.1. The functionalized liposomes of item 1, wherein the aptamer is selected from a DNA, an RNA or an XNA;
4.2. The functionalized liposomes of item 1, wherein the aptamer is a DNA or an RNA comprising 18-60 nucleotides, such as 20-25 nucleotides; and/or
4.3. The functionalized liposomes of item 1, wherein the K_{d} of the aptamer to its target in or on cornea is about 0.1 nM to 300 nM.
5. The functionalized liposomes of item 1, wherein the aptamer is a mucin-targeting aptamer.
6.1. The functionalized liposomes of item 5, wherein the mucin-targeting-aptamer comprises a nucleotide sequence of SEQ ID NO: 1.
6.2. The functionalized liposomes of item 5, wherein the K_{d} of the mucin-targeting aptamer to mucin-1 in or on cornea/cornea epithelial cells is about 0.1 nM to 50 nM.
7.1. The functionalized liposomes of item 1, wherein the aptamer is conjugated to or inserted into the outer surface of the liposome.
7.2. The functionalized liposomes of item 1, wherein the number of aptamer on each liposome is about 10˜500.
8. The functionalized liposomes of item 1, wherein the eye disease is a noncancerous disease.
9. The functionalized liposomes of item 1, wherein the active ingredient is one or more selected from the group consisting of: an active ingredient for preventing or treating dry eye disease (DED), glaucoma, cataract, keratitis, conjunctivitis, mydriasis, optic neuritis, diabetic retinopathy, refractive error, fundus hemorrhage, strabismus, night blindness, macular degeneration, pterygium, ocular calculi, lacrimal sac.
10. The functionalized liposomes of item 1, wherein the active ingredient for preventing and/or treating eye disease is one or more selected from the group consisting of: a polypeptide agent, an anti-glaucomatous agent, an antibiotics, an antibacterial agent, an antiviral agent, an anti-inflammation agent, or any combinations thereof.
11. The functionalized liposomes of item 10, wherein the polypeptide agent is cyclosporine A, defensins, interferons, or epidermal growth factor; and/or
wherein the anti-glaucomatous agent is β-blockers; and/or
wherein the antibiotics is β-lactam, aminoglycosides, or macrolide; and/or
wherein the antibacterial agent is quinolone or imidazole; and/or
wherein the antiviral agent is antiviral nucleosides; and/or
wherein the anti-inflammation agent is steroidal anti-inflammatory drugs or non-steroidal anti-inflammatory drugs.
12.1. The functionalized liposomes of item 1, wherein the percentage loading content of the active ingredient in the liposome is about 0.001˜40 wt % based on the total weight of the liposome.
12.2 The functionalized liposomes of item 1, wherein the encapsulation efficiency of the active ingredient in the liposome is about 0.1˜100%.
13. The functionalized liposomes of item 1, wherein the active ingredient trapped in the bilayer region of the liposome, or is encapsulated in the liposome.
14. A product for preventing and/or treating eye disease comprising the aptamer functionalized liposomes loaded with active ingredient(s) for preventing and/or treating eye disease, and a pharmaceutically or physiological acceptable carrier and/or excipient.
15. The product of item 14, wherein the product is in a form of eye drop, eye ointment, eye cream, eye gel, eye mask, eye injection, contact lens.
16. The product of item 14, wherein the pharmaceutically or physiological acceptable carrier and/or excipient is one or more selected from sterile water, buffer, dispersion media, stabilizer, preservative, surfactant.

In items 14-16, the aptamer functionalized liposomes loaded with active ingredient(s) are selected from any one of items 1-13.
17. A method for preventing and/or treating eye disease in a subject in need of the prevention and/or treatment comprising administering an effective amount of a aptamer functionalized liposomes loaded with active ingredient(s) for preventing and/or treating eye disease or a product comprising the same to the eye(s) of the subject.

In item 17, the aptamer functionalized liposomes loaded with active ingredient(s) are selected from any one of items 1-13, and the product is selected from any one of items 14-16.
18. A method for targeted drug delivery to the corneal epithelium of a subject in need thereof comprising administering an effective amount of a aptamer functionalized liposomes loaded with active ingredient(s) for preventing and/or treating eye disease or a product comprising the same to the eye(s) of the subject.

In item 18, the aptamer functionalized liposomes loaded with active ingredient(s) are selected from any one of items 1-13, and the product is selected from any one of items 14-16.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

It is preferred to select and combine preferred embodiments described herein and the specific subject-matter arising from a respective combination of preferred embodiments also belongs to the present disclosure.

### EXAMPLES

The present invention is further illustrated by the following examples. These examples are provided merely for illustration purposes and shall not be interpreted to limit the scope or content of the present invention in any way.

Publications cited herein and the materials for which they are cited are hereby specifically incorporated by reference in their entireties. All reagents, unless otherwise indicated, were obtained commercially. All parts and percentages are by weight unless stated otherwise. An average of results is presented unless otherwise stated. The abbreviations used herein are conventional, unless otherwise defined.

Materials and Methods

### 1. Materials

Phospholipids were purchased from Avanti Polar Lipids. All aptamers and primers were purchased from Integrated DNA Technologies. Cyclosporine, MTT, and Fluoromount™ Aqueous Mounting Medium were purchased from Sigma-Aldrich. Amicon Ultra-0.5 mL Centrifugal Filters were purchased from Millipore Sigma. All the cell culture-related chemicals including medium, serum, and antibiotics were purchased from Fisher Scientific Inc. Culture flasks and Hoechst 33342 solution were purchased from Thermo Fisher. Sodium chloride, Isol-RNA Lysis Reagent were purchased from VWR. PBS, iScript™ cDNA Synthesis Kit, and SsoFast EvaGreen Supermixes were purchased from Bio-Rad. Coverslips were purchased from SPL Life Sciences. Milli-Q water was used to prepare all buffers, solutions, and suspensions.

### 2. Preparation of CsA-Loaded Liposome

Liposomes were prepared using the standard extrusion method. DOPC (1,2-dioleoyl-sn-glycero-3-phosphocholine) and Rhod PE (1,2-dioleoyl-sn-glycero-3-phosphoethanolamine-N-(lissamine rhodamine B sulfonyl)) (ammonium salt) at a weight ratio of 99:1 were dissolved in chloroform with a total lipid mass of 2.5 mg. CsA in chloroform were mixed into the lipid mixture at various weight percentages (0, 2, 5, 10, 20, 40, 50, 100 wt % of lipid). The chloroform was removed by blowing with N₂ followed by drying in a vacuum oven overnight at RT. The lipid thin films were then rehydrated and dissolved in 0.5 mL PBS at RT with sonication. The lipid concentration after rehydration was 5 mg/ml. The lipid suspensions were subjected to extrusion with two stacked polycarbonate membranes (100 nm pore size) and two syringes for 11 times at RT. The liposome suspensions were stored at 4° C. until use.

### 3. Dynamic Light Scattering

20 μL of the freshly extruded liposome samples were diluted 50 times with PBS. The hydrodynamic diameter and zeta potential of the liposome were determined by dynamic light scattering (Zetasixer Nano, Malvern).

### 4. CsA Quantification by UV-Vis Spectrophotometer

The measurement of CsA in liposomes was performed by UV-vis spectroscopy (Dell Optiplex GX1) at a fixed wavelength of 205 nm. Serial concentrations of CsA dissolved in buffer A (PBS: Ethanol at the ratio of 1:10) were used to construct a standard curve for CsA quantification. To measure the total CsA in liposome suspension, samples were suspended in ethanol at a 1:10 ratio (sample:ethanol) and vortex for 1 minute to destroy the liposome structure, releasing the loaded CsA into the solution. The samples were further diluted 10 times with buffer A before UV-vis analysis. To measure the unloaded CsA in the liposome suspension, unloaded CsA was separated by 3K Amicon Ultra-0.5 mL Centrifugal Filters (14,000 rpm, 10 minutes). 20 μL filtrates were diluted with 200 μL ethanol before UV-vis measurement. The CsA content in samples was determined by subtracting the absorbance of liposome alone from that of CsA-loaded liposome and compared with the standard curve.

### 5. Determination of Encapsulation Efficiency and Drug Loading

The encapsulation efficiency and drug-loading content of the CsA in liposomes were calculated according to the following equations:

$$Drug loading content ( \% ) = \frac{Weight of CsA in Liposome}{Weight of liposome} \times 100$$

$$Encapsulation efficiency ( \% ) = \frac{Weight of total CsA - weight of unloaded CsA}{Weight of initial CsA} \times 100$$

### 6. Aptamer Conjugation and Analysis

S2.2 is a cholesterol linked 25-base mucin-1 aptamer with relatively high affinity and specificity to MUC-1.^{[9]}

S2.2: 5′-GCAGTTGATCCTTTGGATACCCTGGTTTTT-Chol-3′.

To conjugate the S2.2 aptamer with liposome or CsA-loaded liposome, 20 μL liposome suspension (5 mg/mL) was mixed with 10 μL aptamer (100 μM) in 20 μL PBS and 20 μL NaCl (pH 7.5, 5 M) and MilliQ water to achieve a final volume of 200 μL. After overnight incubation, the free aptamer was separated from the liposome by ultracentrifugation (Beckman Optima TLX Ultracentrifuge) at 120,000 rpm for 30 minutes. The concentrations of free aptamer were measured by spectrophotometer (Tecan Spark). The liposome pellets were resuspended with 20 μL PBS (Liposome concentration at 5 mg/ml). A cholesterol linked non-aptamer, B2, was conjugated with the CsA-loaded liposome as a negative control:

B2: 5′-ACTTCAACATCTAGCTGGTGG-Chol-3′.

### 7. Human Corneal Epithelial Cell (HCEC) Culture and Hyperosmotic Stress Induction

HPV-immortalized HCECs were cultured based on Dr. Gorbet's method with a few modifications. The cells (passage 4-9) were maintained in DMEM/F12 medium supplemented with 1% FBS and 1% penicillin/streptomycin at 37° C., 95% humidity and 5% CO2. The cell medium was changed every 2 days. To establish an in vitro DED model, sodium chloride was added to the culture medium to increase the osmolarity of the medium by 200 mOsm.

### 8. Cell Viability

HCECs were seeded in 96-well plates at 12,000 cells/well and incubated for 24 hours. Afterward, HCECs were treated with vehicle (phosphate buffered saline), CsA (0.002%, 0.001% and 0.0001% in medium), drug-free liposomes or CsA-loaded liposomes (0.5, 5, 50, 100 μg/mL liposomes) under serum-free hyperosmolarity medium for 24 hours. HCECs cultured in the isosmotic medium with vehicle were used as a control. Cells were then incubated in 0.5 mg/ml MTS for 4 hours. The medium was discarded and 100 μl of DMSO was added to each well to dissolve the precipitates. The absorbance was detected at 570 nm with a Microplate Reader (Tecan Spark).

### 9. Cellular Uptake

For 4-hour cellular uptake, HCECs were seeded on cell culture-grade coverslips in 48-well plates at 25,000 cells/well for 24 hours. Afterward, HCECs were treated with B2-conjugated CsA-loaded liposomes, S2.2-conjugated drug-free liposomes, or S2.2-conjugated CsA-loaded liposomes (5 μg/mL liposomes) under serum-free hyperosmolarity medium for 4 hours.

To test the retention of liposomes in cells, HCECs were treated with liposomes for 4 hours as described. It was followed by 20-hour incubation in the hyperosmolarity medium. HCECs cultured in the isosmotic medium were used as a control.

After 4- or 24-hour incubation, the cells on coverslips were then washed 2 times with ice-cold PBS and fixed with fresh 4% paraformaldehyde for 15 minutes at RT. The cells were then stained with 1 μg/mL Hoechst 33342 in PBS for nucleus staining for 1 minute at RT. The coverslips were mounted on glass microscope slides with a drop of Fluoromount™ Aqueous Mounting Medium to reduce fluorescence photobleaching and sealed with nail polish. Fluorescence images were captured at the mid-plane of cells (magnification: 400×) by Nikon Eclipse Ti Inverted Research Microscope. The excitation wavelength of Hoechst and Rhod PE were 361 nm and 560 nm, respectively.

### 10. Real-Time PCR

HCECs were seeded in 6-well plates at 250,000 cells/well and incubated for 24 hours. Afterward, HCECs were treated with CsA (0.001% and 0.0001% in medium), B2-conjugated CsA-loaded liposomes, S2.2-conjugated drug-free liposomes, or S2.2-conjugated CsA-loaded liposomes (0.5 and 5 μg/mL liposomes) under serum-free hyperosmolarity medium for 4 hours. The cells were washed 2 times with PBS afterward to remove unbound liposomes and CsA. It was followed by 20-hour incubation in the hyperosmolarity medium. HCECs cultured in the isosmotic medium with vehicle were used as a control. 24 hours after cell seeding, RNA was extracted from cells using Isol-RNA Lysis Reagent and undergone reverse transcription using iScript™ cDNA Synthesis Kit following the manufacturer's instruction. The gene expressions of target genes, including interleukin-1β (IL-1B), interleukin-6 (IL-6), interleukin-8 (IL-8), matrix metalloproteinase-1 (MMP-1), glyceraldehyde-3-phosphate dehydrogenase (GAPDH) in treated cells, were measured using SsoFast EvaGreen with specific primer pairs. The gene expressions were normalized with GAPDH. Primers used are shown in Table 2 (SEQ ID NOs: 3-12).

### 11. Cell Membrane Integrity

HCECs were seeded on cell culture-grade coverslips in 48-well plates at 25,000 cells/well for 24 hours. The cells were treated as mentioned in section 10. The medium was aspirated and the cells were rinsed 2 times with PBS. The membrane integrity was studied using fluorescein uptake assay. After incubation of fluorescein (1 mM in medium) for 5 minutes, the solution was discarded and the cells were further washed with PBS. The fluorescein uptake was captured as mentioned in section 9. The excitation wavelength was 498 nm. The overall intensities of the fluorescent signals were also quantified using the corresponding Nikon system.

### 12. Statistical Analysis

For MTT, RT-PCR, and cell cellular uptake assays, the data were reported as mean±SEM. Inter-group differences were analyzed by one-way ANOVA with Tukey's as a post hoc test. A value of P<0.05 was considered statically significant. All graphs in this study were plotted by using GraphPad Prism Version 9.0. Three independent trails were conducted for in vitro study.

### Example 1. Characterization of the Synthesized Liposomes

The lipid composition of liposomes determines their physiochemical properties and assists drug delivery, which has already had a major impact on many biomedical areas.^{[11]} Using partial lipid composition of the cell membrane is a widely used strategy for enhancing drug delivery and fusion with the cell membrane.^{[11, 12]} We adopted DOPC in the present study because it is the dominant component of the phospholipid bilayer of cell membranes. In addition, 1% of rhodamine-labelled PE (Rhod PE) was used as an indicator to visualize liposomes in cells.

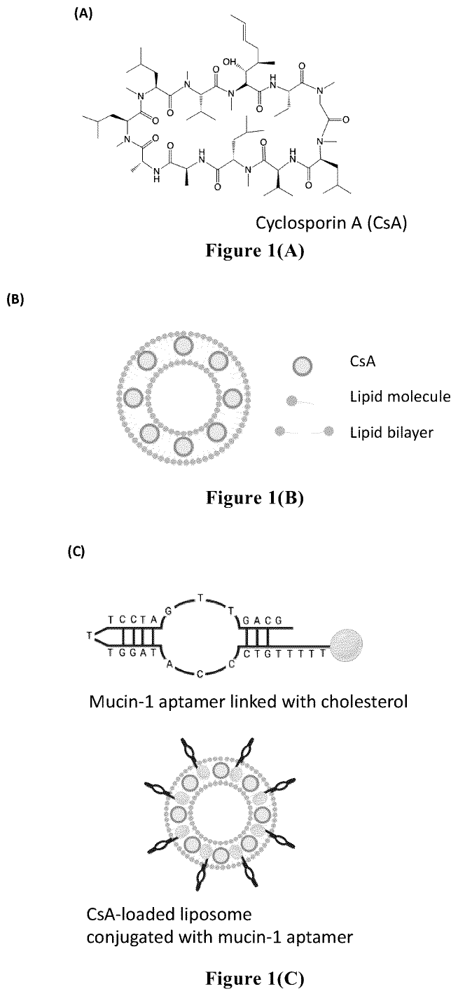

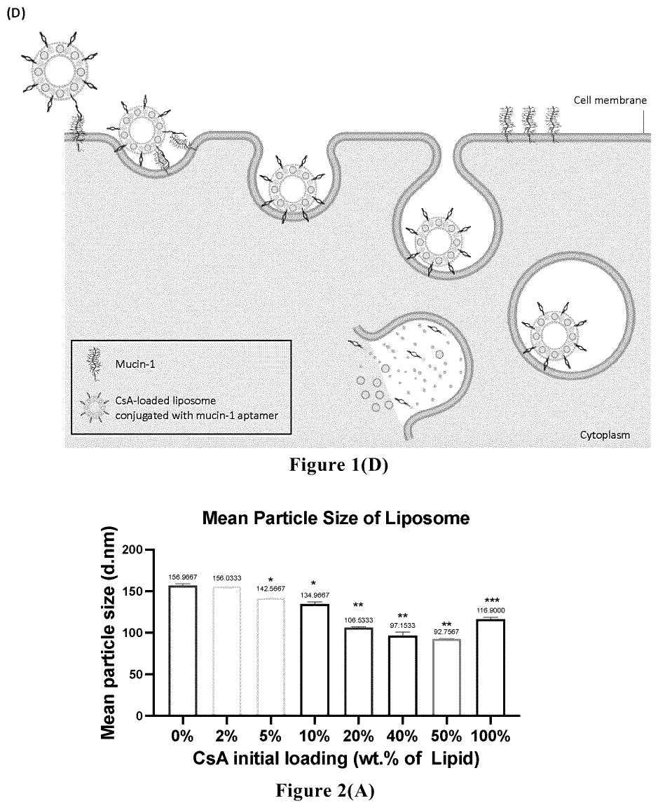

CsA is a hydrophobic molecule which could be trapped in the bilayer region of liposomes (FIG. 1(A)). To optimize the best loading condition, we co-dissolved the lipids and CsA at 2, 5, 10, 20, 40, 50, 100 wt. % of lipid in chloroform, which was then subjected to thin-film hydration and 11-round extrusion to achieve a narrow-size distribution of liposomes close to the size of membrane pore (100 nm). Since CsA is a hydrophobic molecule, it should be in the bilayer region of the liposomes (FIG. 1(B)). We analyzed the size and zeta potential of liposomes using DLS.

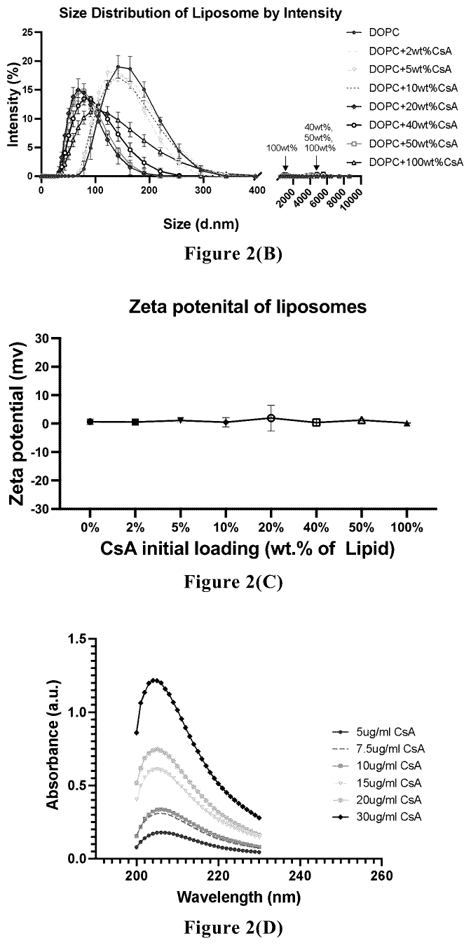

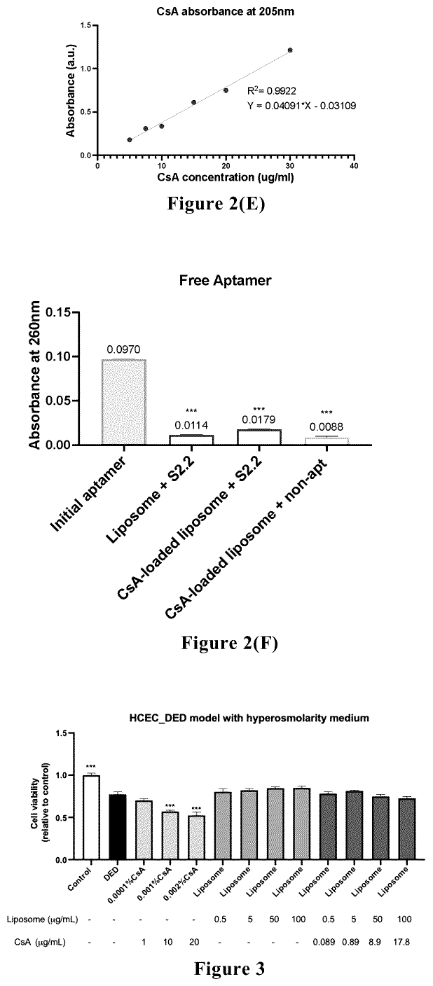

The results in FIG. 2(A) showed that the unloaded liposomes have a mean particle size of 157 nm. The mean size of liposome decreased with the increase of CsA loading from 2 wt % (156 nm) to 50 wt % (93 nm). Apart from mean particle size, the size distribution profile (FIG. 2(B)) suggested that there had narrow size distributions, deviation within 200 nm, in the liposome suspension loading with 2, 5, 10, and 20 wt. % CsA. However, large aggregations were observed in liposome suspension loaded with 40, 50, and 100 wt % CsA. The particle sizes of these aggregations were around 1500 nm (40, 50, 100 wt % CsA) and 5000 nm (100 wt % CsA).

In general, liposomes can be classified into three types by sizes: small unilamellar vesicles (15-100 nm in diameter), large unilamellar vesicles (100-1000 nm in diameter), and giant unilamellar vesicles (1-5 μm in diameter). Small unilamellar vesicles and large unilamellar vesicles have a larger specific surface area which might increase solubility, enhance the bioavailability, and enable accurate targeting of the encapsulated material to a greater extent.^{[12]} Therefore, the 10, 20 wt % CsA groups were chosen for the subsequent assays, while the 40, 50 wt % CsA groups were chosen to compare the encapsulation efficacy to 10, 20 wt % CsA groups. No significant changes were observed in the zeta potential of liposomes in all samples (FIG. 2(C)).

We used UV-vis spectroscopy to quantify the CsA in samples. CsA had a peak UV absorbance at around 210 nm when dissolved in ethanol.^{[13]} In the present study, we constructed a calibration curve using PBS: ethanol at 1:10 ratio as the solvent because the liposomes were suspended in PBS and we used ethanol to break the liposomes. Based on the UV spectra and calibration curve (FIG. 2(D) and FIG. 2(E)), CsA had a peak absorbance at 205 nm when it was dissolved in the PBS: ethanol 1:10 solution. The R² value of the calibration curve is 0.9922. After subtracting the background absorbance in drug-free liposomes and comparing the UV absorbance of samples to the calibration curve, the total CsA and unloaded CsA in four liposome suspensions were determined (Table 1). The total CsA contents in the 10, 20, 40 and 50 wt % samples were 503, 895, 605 and 772 g/ml while the unloaded CsA contents in 10, 20, 40 and 50 wt % samples were 10.1, 17.1, 23.8 and 29.9 μg/ml, respectively. The drug loading content and encapsulation efficiency were also calculated (Table 1). In general, an optimum loading condition could achieve an entrapment efficiency of around 90% or more.^{[14]} Although the 10 wt % CsA group had over 90% encapsulation efficiency, the 20 wt % CsA group had the highest CsA content (17.56%) with a comparable encapsulation efficiency (87.8%). Therefore, the 20 wt % CsA group was subjected to aptamer conjugation and biological study.

TABLE 1
Quantification of CsA in liposome

| | | | |
|---|---|---|---|
| 10 wt. % | 20 wt. % | 40 wt. % | 50 wt. % |

Total CsA

| | | | | |
|---|---|---|---|---|
| Absorbance at 205 nm | 0.16 | 0.30 | 0.19 | 0.26 |
| [CsA loaded lipsome-liposome] | | | | |
| Total CsA (μg/mL) in liposome (5 mg/mL) | 502.7 | 895.4 | 605 | 772.2 |

Unloaded CsA

| | | | | |
|---|---|---|---|---|
| Absorbance at 205 nm | 0.007 | 0.033 | 0.057 | 0.080 |
| [CsA loaded lipsome-liposome] | | | | |
| Unloaded CsA (μg/mL) in liposome (5 mg/mL) | 10.12 | 17.05 | 23.76 | 29.92 |

Drug loading content (%)

| | | | | |
|---|---|---|---|---|
| (Weight of CsA in Liposome/ | 9.85 | 17.56 | 11.62 | 14.84 |
| Weight of liposome) × 100 | | | | |

Encapsulation efficiency (%)

| | | | | |
|---|---|---|---|---|
| (Weight of total CsA-weight of unloaded CsA)/Weight of | 97.51 | 87.83 | 29.06 | 26.69 |
| initial CsA) × 100 | | | | |

### Example 2. Characterization of the CsA-Loaded Liposome Conjugated with Mucin-1 Aptamer

The S2.2 mucin-1 aptamer reported by the Ferreira group was adopted in the present study.^{[9]} It has a K_{d} value of 0.13 μM toward mucin-1. We also confirmed its specificity to mucin 1 in MCF-7 cells with a mucin 1 knockdown cell line.

Mucin-1 is a membrane-associated heavy molecular glycoprotein that is responsible for lubricating the ocular surface to facilitate smooth blinking, formatting a smooth spherical surface for vision, providing an ocular barrier, and trapping and removing pathogens and debris. Recent research demonstrated that current formulated drugs with micro and nanoparticles were rapidly cleared by ocular mucus.^{[15]} Also, Caffery and co-workers revealed that mucin-1 proteins in treat film were increased in patients with Sjogren's DED.^{[16]} Thus, we chose mucin-1 as the delivery target to reduce the clearance by mucus, especially under dry eye conditions.

To investigate the effect of the S2.2 aptamer on liposome retention time, we employed a non-aptamer control sequence. Both sequences were conjugated with cholesterol for insertion into liposomes (FIG. 1(C)). After ultracentrifugation, the free aptamers were measured by spectrophotometer. The results from FIG. 2(F) suggested around 80-90% of the initial aptamers were attached to the liposomes. We estimated the number of DNA strands on each liposome to be 344.

### Example 3. Cytotoxicity, Cellular Uptake and Retention Time of Synthesized Liposomes in In Vitro Dry Eye Model

For the in vitro study, we constructed a dry eye model in HCECs by increasing the osmolarity of the medium. Raising the osmolarity of the medium by adding sodium chloride has been reported to induce cell death and inflammatory marker expression in HCECs, mimicking the situation in DED.^{[17, 18]} Previous studies showed that 0.05% CsA could induce cell death in HCECs in 10 min. Thus, we first studied the cytotoxicity of CsA-loaded liposomes. As expected, the results in FIG. 3 suggested that increasing osmolarity of the medium reduced the cell viability of HCECs compared to the control group, while treatment of 0.001% and 0.002% CsA in the medium for 24 h remarkably suppressed the viability of HCECs compared to DED groups. It implied the cytotoxicity of CsA alone in HCECs. Interestingly, neither the drug-free liposomes nor the CsA-loaded liposomes at all dosages induced cell death in the DED model, suggesting the encapsulation of CsA in liposomes could lessen the cytotoxic effects of CsA in HCECs.

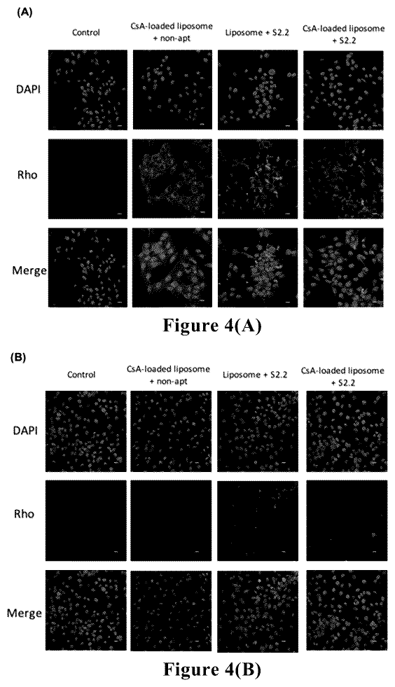

We then investigated the cellular uptake of liposomes in HCECs. The confocal images in FIG. 4(A) suggested that some liposome were found saturated in the cytoplasm after 4 h after washing out those unbound. Nanoparticles including liposomes, when coated with a dense layer of DNA, are called spherical nucleic acids, which can readily enter cells. A study reported that nanoparticle conjugated with spherical nucleic acid could be internalized by cells through class A scavenger receptors and endocytosed via lipid-raft-dependent, caveolae-mediated manner.^{[20]} In particular, there were observable dense clumps of fluorescence signals in the samples incubated with S2.2 aptamer conjugated liposomes. It might suggest that S2.2 aptamer could increase the attachment and interaction of liposomes to the cell surface (FIG. 1(D)).

We further studied the retention time of liposomes with S2.2 or non-aptamer conjugation in HCECs by prolonging the incubation time to 24 h in total after washing out the unbound liposome in 4 h. The results in FIG. 4(B) indicated that there were no observable rhodamine signals in both control and CsA-loaded liposomes conjugated with non-aptamer. However, there were remarkable rhodamine signals, although weaker than those signals in 4 h, in HCECs treated with both drug-free and CsA-loaded liposomes conjugated with S2.2 aptamer. These results supported that the high affinity and mucin-1-specific aptamer increased the retention of liposomes in cells.

### Example 4. Efficacy of Aptamer Functionalized Liposomes Loaded with CsA on In Vitro Dry Eye Model

The efficacy of aptamer functionalized liposome on dry eye model was investigated. We measured the gene expressions of three inflammatory markers (IL-1, IL-8, IL-β) and a matrix metalloprotease (MPP-1) in HCECs. These markers were reported to be upregulated in tears in patients with DED and were down-regulated by CsA in both in vivo and in vitro studies.^{[21]}

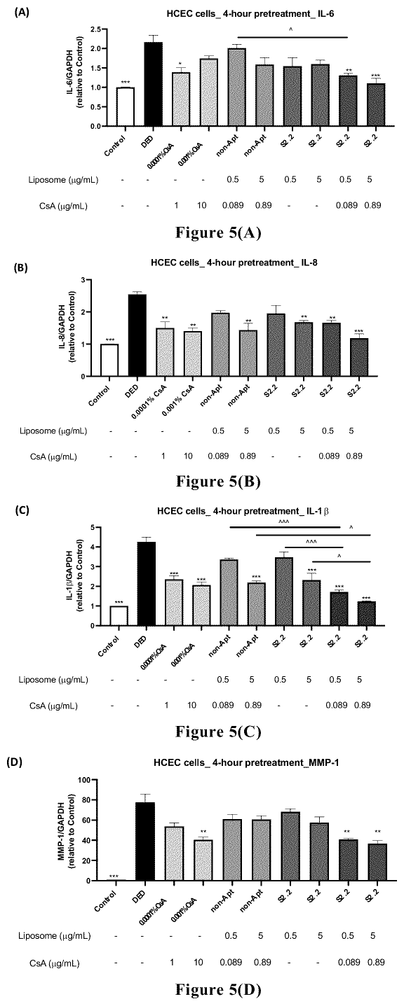

In FIGS. 5(A)-5(C), the gene expressions of three inflammatory markers were increased significantly by hyperosmotic pressure for 24 h compared to control groups. The pre-treatment of CsA dramatically reversed the expression of IL-8, IL-1β at 1 and 10 μg/ml, and IL-6 at 1 μg/ml. The pre-treatment of CsA-loaded liposome conjugated with non-aptamer or drug-free liposome conjugated with S2.2 aptamer at low doses did not exert any anti-inflammatory effect in the DED model in HCECs. Only the treatment of high-dose liposomes containing 0.89 μg/ml CsA could suppress the gene expression of IL-8, IL-1β induced by hyperosmolarity in HCECs. As for CsA-loaded liposomes conjugated with S2.2 aptamer, it could notably reverse the gene expressions of all inflammatory markers at both doses in DED HCECs. Most of all, there was no statistical significance between the groups treated with CsA-loaded liposome conjugated with S2.2 aptamer and CsA alone, suggesting their effects were comparable. Also, there were significant changes in IL-6 or IL-1B regulation comparing the groups treated with CsA-loaded liposome conjugated with S2.2 aptamer to the non-aptamer liposome or drug-free liposome group, indicating the effect of the CsA in liposome was more potent when it was delivered with the mucin-1 aptamer.

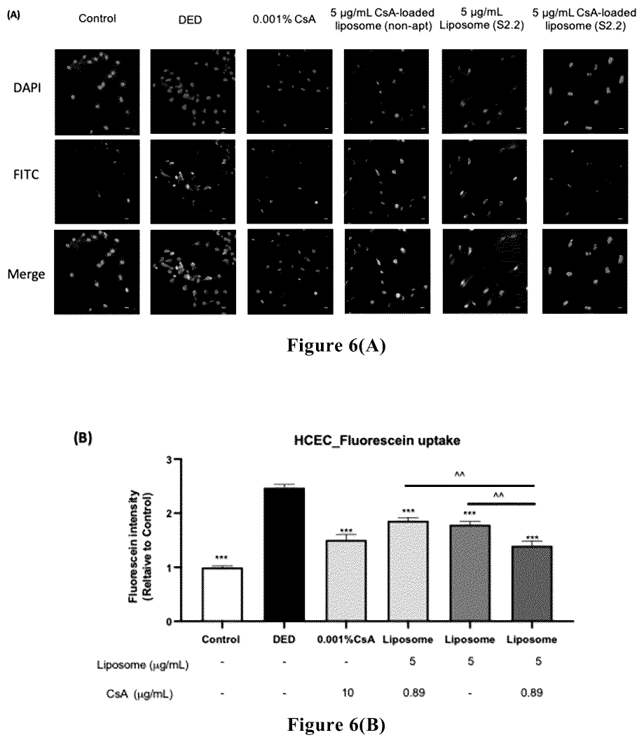

Tight junction between adjoining cells is one of the barriers in the eyes to prevent external substances from entering the eyes and maintaining tear film structure. In dry eye patients, there is an increase in matrix metalloproteinase, including collagenase (MMP-1, MMP-13) and gelatinases (MMP-9) in tears which are responsible for corneal epithelium disruption.^{[22, 23]} It was reported that hyperosmotic stress could induce MMP expression and break tight junctions. It was also supported by the data in FIG. 5(D) and FIG. 6. The gene expression of MMP-1 increased intensively in HCECs in a hyperosmolarity medium. Also, the uptake of fluorescein were increased in the DED model.^{[24-26]} We also showed similar results in dye uptake in the DED model compared to the control groups (FIG. 6(A)). For gene expression, only free CsA and CsA-loaded liposome conjugated with S2.2 aptamer could reverse the MMP-1 expression in HCECs. On the other hand, all treatments appear to reduce the uptake of both dyes, indicating all treatments were effective in regulating the tight junction and cell permeability of HCECs under dry eye conditions. In particular, there were significant differences in the fluorescent intensity between CsA-loaded liposome conjugated with the S2.2 aptamer group and the other two liposome groups (FIG. 6(B)). These results indicated the presence of S2.2 aptamer and CsA in the liposome provided greater protection in HCECs in regards of cell permeability.

It was believed that the drug-free liposome at higher doses worked in inhibiting the inflammation and description of tight junction in HCECs because restoring the lipid content in corneal epithelium to prevent inflammation, evaporation of aqueous in the eye, and subsequent drying is one of the therapeutic approaches of dry eye disease and corneal injury.^{[27]} The presence of liposomes might contribute to the action of CsA-loaded liposomes conjugated with S2.2 aptamer in anti-inflammation in vitro. However, the results surprisingly show that CsA at lower doses (0.089 μg/ml) in liposome conjugated with S2.2 aptamer could achieve comparable effect of CsA alone (1 and 10 μg/ml) in HCECs. In addition, CsA at lower doses in liposome conjugated with S2.2 aptamer showed remarkably improved effect as compared to effect of CsA alone at much higher doses and effect of CsA-loaded liposomes conjugated with non-apt.

### CONCLUSIONS

To our understanding, it is the first research studying the use of aptamer-functionalized liposomes for the delivery of drug to the corneal epithelium. Our data demonstrated that the CsA-loaded liposome conjugated with S2.2 had better retention time and protective effect in HCECs under dry eye conditions. Hence, the present application brings a better tool for ocular drug delivery to treat eye diseases.

The present invention is not to be limited in scope by the embodiments disclosed herein, which are intended as single illustrations of individual aspects of the invention, and any that are functionally equivalent are within the scope of the invention. Various modifications to the compositions and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE 2
Information of the Sequence Listing

| SEQ | | |
|---|---|---|
| ID | | |
| NO: | INFORMATION | SEQUENCE |
| 1 | Nucleotide sequence | GCAGTTGATCCTTTGGATACCCTGG |
| | of Aptamer in S2.2 | TTTTT |
| 2 | Nucleotide sequence | ACTTCAACATCTAGCTGGTGG |
| | of Non-Aptamer in B2 | |
| 3 | IL-6 Forward Primer | CACAGACAGCCACTCACCTC |
| 4 | IL-6 Reverse Primer | TTTTCTGCCAGTGCCTCTTT |
| 5 | IL-8 Forward Primer | TTTCAGAGACAGCAGAGCACACAA |
| 6 | IL-8 Reverse Primer | CACACAGAGCTGCAGAAATCAGG |
| 7 | MMP-1 Forward Primer | GGAGGGGATGCTCATTTTGATG |
| 8 | MMP-1 Reverse Primer | TAGGGAAGCCAAAGGAGCTGT |
| 9 | IL-1β Forward Primer | CCTGTCCTGCGTGTTGAAAGA |
| 10 | IL-1β Reverse Primer | GGGAACTGGGCAGACTCAAA |
| 11 | GAPDH Forward Primer | GAAGGTGAAGGTCGGAGTC |
| 12 | GAPDH Reverse Primer | GAAGATGGTGATGGGATTTC |

What is claimed is:

1. Aptamer functionalized liposomes loaded with active ingredient(s) for preventing and/or treating eye disease.

2. The functionalized liposomes of claim 1, wherein the mean particle size of the liposomes is about 10˜1000 nm (such as 50˜500 nm, 60˜200 nm); and/or
wherein the zeta potential of the liposome is about 0˜5 mV; and/or
wherein the liposomes are prepared from lipids have an anionic, cationic or zwitterionic hydrophilic head group.

3. The functionalized liposomes of claim 1, wherein the liposomes are prepared from phospholipid selected from phosphatidylethanolamines (PE), phosphatidylcholines, phosphatidylserines, and phosphatidyl-glycerols, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), DPPC, DSPC, DOPC, dodecylphosphocholine, 1,2-dioleoyl-sn-glycero-3-phosphatidylethanolamine (DOPE), and 1,2-diphytanoyl-sn-glycero-3-phosphoethanolamine (DPyPE).

4. The functionalized liposomes of claim 1, wherein the aptamer is selected from a DNA, an RNA or an XNA; and/or
wherein the aptamer is a DNA or an RNA comprising 18-60 nucleotides, such as 20-25 nucleotides; and/or
wherein the K_{d} of the aptamer to its target in or on cornea is about 0.1 nM to 300 nM.

5. The functionalized liposomes of claim 1, wherein the aptamer is a mucin-targeting aptamer.

6. The functionalized liposomes of claim 5, wherein the mucin-targeting-aptamer comprises a nucleotide sequence of SEQ ID NO: 1; and/or
wherein the K_{d} of the mucin-targeting aptamer to mucin-1 in or on cornea/cornea epithelial cells is about 0.1 nM to 50 nM.

7. The functionalized liposomes of claim 1, wherein the aptamer is conjugated to or inserted into the outer surface of the liposome; and/or
wherein the number of aptamer on each liposome is about 10˜500.

8. The functionalized liposomes of claim 1, wherein the eye disease is a noncancerous disease.

9. The functionalized liposomes of claim 1, wherein the active ingredient is one or more selected from the group consisting of: an active ingredient for preventing or treating dry eye disease (DED), glaucoma, cataract, keratitis, conjunctivitis, mydriasis, optic neuritis, diabetic retinopathy, refractive error, fundus hemorrhage, strabismus, night blindness, macular degeneration, pterygium, ocular calculi, lacrimal sac.

10. The functionalized liposomes of claim 1, wherein the active ingredient for preventing and/or treating eye disease is one or more selected from the group consisting of: a polypeptide agent, an anti-glaucomatous agent, an antibiotics, an antibacterial agent, an antiviral agent, an anti-inflammation agent, or any combinations thereof.

11. The functionalized liposomes of claim 10, wherein the polypeptide agent is cyclosporine A, defensins, interferons, or epidermal growth factor; and/or
wherein the anti-glaucomatous agent is β-blockers; and/or
wherein the antibiotics is β-lactam, aminoglycosides, or macrolide; and/or
wherein the antibacterial agent is quinolone or imidazole; and/or
wherein the antiviral agent is antiviral nucleosides; and/or
wherein the anti-inflammation agent is steroidal anti-inflammatory drugs or non-steroidal anti-inflammatory drugs.

12. The functionalized liposomes of claim 1, wherein the percentage loading content of the active ingredient in the liposome is about 0.001˜40 wt % based on the total weight of the liposome; and/or
wherein the encapsulation efficiency of the active ingredient in the liposome is about 0.1˜100%.

13. The functionalized liposomes of claim 1, wherein the active ingredient trapped in the bilayer region of the liposome, or is encapsulated in the liposome.

14. A product for preventing and/or treating eye disease comprising the aptamer functionalized liposomes loaded with active ingredient(s) for preventing and/or treating eye disease, and a pharmaceutically or physiological acceptable carrier and/or excipient.

15. The product of claim 14, wherein the product is in a form of eye drop, eye ointment, eye cream, eye gel, eye mask, eye injection, contact lens.

16. The product of claim 14, wherein the pharmaceutically or physiological acceptable carrier and/or excipient is one or more selected from sterile water, buffer, dispersion media, stabilizer, preservative, surfactant.

17. A method for preventing and/or treating eye disease in a subject in need of the prevention and/or treatment comprising administering an effective amount of a aptamer functionalized liposomes loaded with active ingredient(s) for preventing and/or treating eye disease or a product comprising the same to the eye(s) of the subject.

18. A method for targeted drug delivery to the corneal epithelium of a subject in need thereof comprising administering an effective amount of a aptamer functionalized liposomes loaded with active ingredient(s) for preventing and/or treating eye disease or a product comprising the same to the eye(s) of the subject.


SEQUENCE LISTING (12 sequences)

SEQ ID NO: 1 - DNA, 30 bp, synthetic construct
  source 1..30: mol_type = other DNA
gcagttgatcctttggataccctggttttt

SEQ ID NO: 2 - DNA, 21 bp, synthetic construct
  source 1..21: mol_type = other DNA
acttcaacatctagctggtgg

SEQ ID NO: 3 - DNA, 20 bp, synthetic construct
  source 1..20: mol_type = other DNA
cacagacagccactcacctc

SEQ ID NO: 4 - DNA, 20 bp, synthetic construct
  source 1..20: mol_type = other DNA
ttttctgccagtgcctcttt

SEQ ID NO: 5 - DNA, 24 bp, synthetic construct
  source 1..24: mol_type = other DNA
tttcagagacagcagagcacacaa

SEQ ID NO: 6 - DNA, 23 bp, synthetic construct
  source 1..23: mol_type = other DNA
cacacagagctgcagaaatcagg

SEQ ID NO: 7 - DNA, 22 bp, synthetic construct
  source 1..22: mol_type = other DNA
ggaggggatgctcattttgatg

SEQ ID NO: 8 - DNA, 21 bp, synthetic construct
  source 1..21: mol_type = other DNA
tagggaagccaaaggagctgt

SEQ ID NO: 9 - DNA, 21 bp, synthetic construct
  source 1..21: mol_type = other DNA
cctgtcctgcgtgttgaaaga

SEQ ID NO: 10 - DNA, 20 bp, synthetic construct
  source 1..20: mol_type = other DNA
gggaactgggcagactcaaa

SEQ ID NO: 11 - DNA, 19 bp, synthetic construct
  source 1..19: mol_type = other DNA
gaaggtgaaggtcggagtc

SEQ ID NO: 12 - DNA, 20 bp, synthetic construct
  source 1..20: mol_type = other DNA
gaagatggtgatgggatttc